(19) United States
(12) Patent Application Publication
(10) Pub. No.: US 2016/0040228 A1
(43) Pub. Date: Feb. 11, 2016

(54) SEQUENCING STRATEGIES FOR GENOMIC REGIONS OF INTEREST

(71) Applicant: Cergentis B.V., Utrecht (NL)

(72) Inventors: Elzo De Wit, Utrecht (NL); Erik Cornelis Splinter, Utrecht (NL)

(21) Appl. No.: 14/829,224

(22) Filed: Aug. 18, 2015

Related U.S. Application Data
(63) Continuation of application No. PCT/NL2014/050101, filed on Feb. 19, 2014.
(60) Provisional application No. 61/766,425, filed on Feb. 19, 2013.

(51) Int. Cl. C12Q 1/68

(52) U.S. Cl. CPC C12Q 1/6874

(57) ABSTRACT

The disclosure relates to methods for determining the sequence of a genomic region of interest comprising a target nucleotide sequence comprising: providing a DNA comprising a genomic region of interest, fragmenting the DNA, separating the DNA fragments, fragmenting and ligating the DNA fragments to provide for ligated fragments of the DNA fragments (or ligated DNA subfragments), and determining at least part of the sequences of at least part of the ligated DNA subfragments that comprise the target nucleotide sequence.

### CROSS-REFERENCE TO RELATED APPLICATIONS

This application is a continuation of International Patent Application Serial No. PCT/NL2014/050101, filed Feb. 19, 2014, and published as International Publication No. WO 2014/129894 A1 on Aug. 28, 2014, pending, which application claims priority to U.S. Provisional Patent Application Ser. No. 61/766,425, filed Feb. 19, 2013, the disclosure of each of which is hereby incorporated herein in its entirety by this reference.

### TECHNICAL FIELD

The present disclosure relates to the field of molecular biology and more, in particular, to DNA technology. The disclosure relates more particularly to the sequencing of DNA. The disclosure relates to strategies for determining (part of) a DNA sequence of a genomic region of interest. The disclosure further relates to uses of the methods of this disclosure in the development of personalized diagnostics and medical treatment and in the screening of tissues for the presence of malignancies and other conditions.

### BACKGROUND

Considerable effort has been devoted to develop “target enrichment” strategies for sequencing, in which genomic regions from a DNA sample are selectively captured and/or selectively amplified and subsequently sequenced (reviewed in Mamanova et al., Nature Methods 2010 (2):111-118). Genomic enrichment strategies are important, as they allow research to focus on a particular genomic region, which, as compared to complete genome analysis, is more time and cost effective, and also much less difficult to analyze. Different genomic enrichment strategies exist. For instance, performing a PCR reaction, using a single primer pair, will amplify a genomic region, and thus enrich for that genomic region. However, the size of PCR product that can be made is limited. Long PCR protocols currently have an upper limit of 10 kB to 40 kB that can be amplified (Cheng et al., Proc. Natl. Acad. Sci. U.S.A. 1994, 91(12):5695-5699), but these approaches tend to lack robustness and each PCR requires optimization and validation, and still, the size limit is restricted. In order to increase the size of regions that can be amplified, as well as the robustness of the assay, tiled approaches have been developed using a multitude of PCR primer pairs designed specifically for a genomic region of interest. These primers are used, for example, in a multiplex PCR approach or a RainDance PCR. Various enzymatic methods, such as target circularization, are compatible with such targeted amplification strategies. Other methods, such as SureSelect by Agilent, involve the use of capture probes, on an array or in solution, wherein probes of 60-120 bases in length are used to capture the genomic region of interest via hybridization.

As is clear from the examples above, in order to enrich a genomic region of interest, sequence information throughout the genomic region of interest is required beforehand, because this is needed to design probes and/or primers to capture and/or amplify the genomic region of interest. For instance, to enrich a 30 Mb sequence, 6,000 separate PCRs would typically be required. With capture probes, even more sequence information is required, as at least as many as 250,000 120 base pair probes would be required and have to be designed to capture a 30 Mb sequence. These assays are biased by using sequence data for the probes and/or primers that largely cover the genomic region of interest. They do not pick up sequences that deviate too much from the designed template sequences and will, therefore, not detect insertions. In addition, these approaches require fragmenting DNA, typically into sequences of a few 100 base pairs before the analysis. This means that the genomic region of interest is broken up into many pieces, resulting in loss of information, a.o., regarding rearrangements within the region of interest. Hence, there is a need for improved genomic enrichment strategies that are much less biased, do not require thousands of short sequences, and enable hypothesis-neutral complete sequencing of the region of interest.

### BRIEF SUMMARY

It was now found that by carrying out the steps, providing a DNA comprising a genomic region of interest, fragmenting the DNA to provide DNA fragments, and separating the DNA fragments, followed by the further fragmenting of the separated DNA fragments to provide for DNA subfragments (i.e., fragmented separated DNA fragments), and ligating the DNA subfragments to obtain ligated DNA subfragments (i.e., ligated fragments of separated DNA fragments), a good starting point is provided for analyzing the genomic region of interest comprising a target nucleotide sequence, i.e., the linear template surrounding a target nucleotide sequence.

When DNA is fragmented from a cell, most DNA fragments will not comprise the target nucleotide sequence. For example, when a target nucleotide, which preferably may be unique, is selected present within a genome, only one DNA fragment per genome will comprise the target nucleotide sequence. A target nucleotide sequence may be unique when a genome is haploid. When DNA fragments are separated, e.g., such that DNA fragments are in separate containers, only one (or two) containers for each cell will comprise a DNA fragment with the target nucleotide sequence. Similarly, when multiple genomes are fragmented, e.g., from multiple cells and/or from a diploid genome, multiple containers will comprise a DNA fragment with the target nucleotide sequence. When each container comprises, on average, a single DNA fragment, and the DNA fragments are subsequently further fragmented to obtain DNA subfragments, and ligated, because the DNA subfragments are contained separately, DNA subfragments carrying the target nucleotide sequence will only be ligated to DNA subfragments that originate from the corresponding DNA fragment.

Hence, DNA subfragments carrying the target nucleotide will only be ligated to DNA subfragments that originate from the DNA fragment comprising the target nucleotide sequence, i.e., being representative for the genomic region of interest. DNA subfragments not originating from the genomic region of interest may not ligate to the DNA subfragment carrying the target nucleotide sequence because they will be not be within the same container. Methods are well known in the art that allow the separation of DNA fragments such that they are held separately and wherein the DNA fragments may be further processed separately.

The ligated DNA subfragments comprising the target nucleotide sequence, and thus the genomic region of interest, may be amplified, i.e., enriched, by using one or more oligonucleotide primer(s) that recognize the target nucleotide sequence. The sequence of the genomic region of interest can subsequently be determined using (high throughput) sequencing technologies well known in the art. The method has little bias, as no extensive sequence information is required to focus on the genomic region of interest. Only, the sequence of the target nucleotide sequence may be required. For instance, a genomic region of interest may comprise an allele of interest. A target nucleotide sequence may be selected such that it is not within the sequence of the allele of interest. A genomic region of interest may then be amplified by using a target nucleotide sequence, without requiring sequence information of the allele of interest other than the primer sequence. Thus, the allele of interest may be enriched for, without requiring further sequence from that allele. The effect is that the method of enrichment is not biased by using oligonucleotides and/or probes that cover the allelic sequence of interest. In addition, as the ligation step involves the ligation of fragments that originate from DNA fragments that are in a separated state, e.g., in separate containers, the method may also allow for the sequence analysis of separate alleles. For instance, when a DNA comprises multiple alleles (e.g., because the DNA sample originates from a heterogeneous cell population, or because the ploidy is greater than one), each allele represents a different linear DNA template. Hence, the method is suitable to determining haplotypes. A DNA subfragment derived from a separated DNA fragment comprising a target nucleotide sequence, may only interact with DNA subfragments that originate from the DNA fragment and thus corresponding allele. Thus, ligated DNA subfragments are representative of the genomic environment from which the DNA subfragments originate. By determining, for example, at least part of the sequences of at least part of the different ligated DNA subfragments, DNA subfragment sequences may be coupled using the sequence information of the different ligated DNA subfragments and a sequence for separate genomic regions of interest may be built.

### DEFINITIONS

In the following description and examples, a number of terms are used. In order to provide a clear and consistent understanding of the specification and claims, including the scope to be given such terms, the following definitions are provided. Unless otherwise defined herein, all technical and scientific terms used have the same meaning as commonly understood by one of ordinary skill in the art to which this disclosure belongs. The disclosures of all publications, patent applications, patents and other references are incorporated herein in their entirety by reference.

Methods of carrying out the conventional techniques used in methods of the disclosure will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al., Molecular Cloning: A Laboratory Manual, 2nd Edition, Cold Spring Harbor Laboratory Press, Cold Spring Harbor, N. Y., 1989; Ausubel et al., Current Protocols in Molecular Biology, John Wiley & Sons, New York, 1987 and periodic updates; and the series Methods in Enzymology, Academic Press, San Diego.

As used herein, the singular forms “a,” “an” and “the” include plural referents unless the context clearly dictates otherwise. For example, a method for isolating “a” DNA molecule, as used above, includes isolating a plurality of molecules (e.g., tens, hundreds, one thousands, tens of thousands, hundreds of thousands, millions, or more molecules).

A “genomic region of interest” according to the disclosure is a DNA sequence of an organism of which it is desirable to determine, at least part of, the DNA sequence. For instance, a genomic region that is suspected of comprising an allele associated with a disease may be a genomic region of interest. As used herein, the term “allele(s)” means any of one or more alternative forms of a gene at a particular locus. In a diploid cell of an organism, alleles of a given gene are located at a specific location, or locus (loci plural) on a chromosome. One allele is present on each chromosome of the pair of homologous chromosomes. Thus, in a diploid cell, two alleles and thus two separate (different) genomic regions of interest may exist.

“Separating DNA fragments” according to the disclosure means that DNA fragments are physically separated. For example, “separating DNA fragments” includes separating portions of the DNA fragments in separate containers. For example, when a DNA of ten megabases is fragmented in DNA fragments of about 100 kilobases, about 100 DNA fragments are generated from the DNA, these can, for instance, be separated and divided over 50 separate containers, each container having on average about two DNA fragments. The 100 DNA fragments can also be divided over 1,000 containers, the result being that the containers having a DNA fragment will stochastically comprise a single DNA fragment. According to the disclosure, “separating DNA fragments” includes any method in which DNA fragments are physically separated and also allow at least the DNA fragments in theirs separated state to be further fragmented in DNA subfragments and subsequently ligated.

A “nucleic acid” according to the present disclosure may include any polymer or oligomer of pyrimidine and purine bases, preferably cytosine, thymine, and uracil, and adenine and guanine, respectively (see Albert L. Lehninger, Principles of Biochemistry, at 793-800 (Worth Pub. 1982), which is herein incorporated by reference in its entirety for all purposes). The present disclosure contemplates any deoxyribonucleotide, ribonucleotide or peptide nucleic acid component, and any chemical variants thereof, such as methylated, hydroxymethylated or glycosylated forms of these bases, and the like. The polymers or oligomers may be heterogeneous or homogenous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

A “sample DNA” is a sample that is obtained from an organism or from a tissue of an organism, or from tissue and/or cell culture, which comprises DNA. A sample DNA from an organism may be obtained from any type of organism, e.g., microorganisms, viruses, plants, fungi, animals, humans and bacteria, or combinations thereof. For example, a tissue sample from a human patient suspected of a bacterial and/or viral infection may comprise human cells, but also viruses and/or bacteria. The sample may comprise cells and/or cell nuclei. The sample DNA may be from a patient or a person who may be at risk or suspected of having a particular disease, for example, cancer or any other condition that warrants the investigation of the DNA of the organism.

“Fragmenting DNA” includes any technique that, when applied to DNA, results in DNA fragments. Techniques well known in the art are sonication, shearing and/or enzymatic restriction, but other techniques can also be envisaged.

“Random fragmentation” according to the disclosure relates to fragmenting methods in which there is no or less control over the sites in the DNA wherein the DNA is cleaved. For example, sonication and shearing are such random fragmentation methods, or enzymatic methods using enzymes like DNase I, which enzyme cleaves DNA at random sites.

A “restriction endonuclease” or “restriction enzyme” is an enzyme that recognizes a specific nucleotide sequence (recognition site) in a double-stranded DNA molecule, and will cleave both strands of the DNA molecule at or near every recognition site, leaving a blunt or a 3′- or 5′-overhanging end. The specific nucleotide sequence that is recognized may determine the frequency of cleaving, e.g., a nucleotide sequence of six nucleotides occurs on average every 4096 nucleotides, whereas a nucleotide sequence of four nucleotides occurs much more frequently, on average every 256 nucleotides.

“Ligating” according to the disclosure involves the joining of separate DNA fragments. The DNA fragments may be blunt ended, or may have compatible overhangs (sticky overhangs) such that the overhangs can hybridize with each other. The joining of the DNA fragments may be enzymatic, with a ligase enzyme, DNA ligase. However, a non-enzymatic ligation may also be used, as long as DNA fragments are joined, i.e., forming a covalent bond. Typically a phosphodiester bond between the hydroxyl and phosphate group of the separate strands is formed.

“Oligonucleotide primers,” in general, refer to strands of nucleotides that can prime the synthesis of DNA. DNA polymerase cannot synthesize DNA de novo without primers. A primer hybridizes to the DNA, i.e., base pairs are formed. Nucleotides that can form base pairs, that are complementary to one another, are, e.g., cytosine and guanine, thymine and adenine, adenine and uracil, guanine and uracil. The complementarity between the primer and the existing DNA strand does not have to be 100%, i.e., not all bases of a primer need to base pair with the existing DNA strand. From the 3′-end of a primer hybridized with the existing DNA strand, nucleotides are incorporated using the existing strand as a template (template directed DNA synthesis). We may refer to the synthetic oligonucleotide molecules that are used in an amplification reaction as “primers.”

“Amplifying” refers to a polynucleotide amplification reaction, namely, a population of polynucleotides that are replicated from one or more starting sequences. Amplifying may refer to a variety of amplification reactions, including but not limited to polymerase chain reaction (PCR), linear polymerase reactions, nucleic acid sequence-based amplification, rolling circle amplification and like reactions.

“Sequencing” refers to determining the order of nucleotides (base sequences) in a nucleic acid sample, e.g., DNA or RNA. Many techniques are available such as Sanger sequencing and High throughput sequencing technologies such as offered by Roche, Illumina and Applied Biosystems.

The term “contig” is used in connection with DNA sequence analysis, and refers to reassembled contiguous stretches of DNA derived from two or more DNA fragments having contiguous nucleotide sequences. Thus, a contig may be a set of overlapping DNA fragments that provides a (partial) contiguous sequence of a genomic region of interest. A contig may also be a set of DNA fragments that, when aligned to a reference sequence, may form a contiguous nucleotide sequence. For example, the term “contig” encompasses a series of (ligated) DNA fragment(s) that are ordered in such a way as to have sequence overlap of each (ligated) DNA fragment(s) with at least one of its neighbors. The linked or coupled (ligated) DNA fragment(s) may be ordered either manually or, preferably, using appropriate computer programs such as FPC, PHRAP, CAP3, etc., and may also be grouped into separate contigs.

An “adaptor” is a short double-stranded oligonucleotide molecule with a limited number of base pairs, e.g., about 10 to about 30 base pairs in length, which are designed such that they can be ligated to the ends of fragments. Adaptors are generally composed of two synthetic oligonucleotides that have nucleotide sequences that are partially complementary to each other. When mixing the two synthetic oligonucleotides in solution under appropriate conditions, they will anneal to each other forming a double-stranded structure. After annealing, one end of the adaptor molecule may be designed such that it is compatible with the end of a restriction fragment and can be ligated thereto; the other end of the adaptor can be designed so that it cannot be ligated, but this does need not to be the case, for instance, when an adaptor is to be ligated in between DNA fragments.

An “identifier” is a short sequence that can be added to an adaptor or a primer or included in its sequence or otherwise used as label to provide a unique identifier. Such a sequence identifier (or tag) can be a unique base sequence of varying but defined length, typically from 4-16 bp used for identifying a specific nucleic acid sample. For instance, 4 bp tags allow 4⁴=256 different tags. Typical examples are ZIP sequences, known in the art as commonly used tags for unique detection by hybridization (Iannone et al., Cytometry 39:131-140, 2000). Identifiers are useful according to the disclosure, as by using such an identifier, the origin of a (PCR) sample can be determined upon further processing. In the case of combining processed products originating from different nucleic acid samples, the different nucleic acid samples may be identified using different identifiers. For instance, as according to the disclosure, sequencing may be performed using high throughput sequencing, multiple samples may be combined. Identifiers may then assist in identifying the sequences corresponding to the different samples. Identifiers may also be included in adaptors for ligation to DNA fragments assisting in DNA fragment sequences identification. Identifiers preferably differ from each other by at least two base pairs and preferably do not contain two identical consecutive bases to prevent misreads. The identifier function can sometimes be combined with other functionalities such as adaptors or primers.

“Size selection,” according to the disclosure, involves techniques with which particular size ranges of molecules, e.g., DNA fragments, ligated DNA subfragments amplified DNA or circularized DNA, are selected. Techniques that can be used are, for instance, gel electrophoresis, size exclusion, gel extraction chromatography, but are not limited thereto, as long as molecules within a particular size range can be selected, such a technique will suffice.

With the term “aligning” and “alignment” is meant the comparison of two or more nucleotide sequence based on the presence of short or long stretches of identical or similar nucleotides. Methods and computer programs for alignment are well known in the art. One computer program that may be used or adapted for aligning is “Align 2,” authored by Genentech, Inc., which was filed with user documentation in the United States Copyright Office, Washington, D.C. 20559, on Dec. 10, 1991.

### BRIEF DESCRIPTION OF THE DRAWINGS

FIG. 1 shows a schematic of a method for determining the sequence of a genomic region of interest according to the disclosure. The method involves:
a DNA is provided comprising a genomic region of interest comprising a target nucleotide sequence
the DNA is fragmented, e.g., by shearing;
the DNA fragments are separated, e.g., by separating the DNA fragments thereby obtaining containers, with each container comprising a DNA fragment;
the separated DNA fragments are subsequently further fragmented in each container, to provide for DNA subfragments;
the DNA subfragments are ligated in the container to provide for ligated DNA subfragments;
the ligated DNA subfragments may be further fragmented and/or ligated and/or pooled, and an amplification step, e.g., PCR, can be performed with an (inverse) PCR primer set for the target nucleotide sequence (also referred to as viewpoint) within the genomic region of interest. DNA subfragments ligated to the DNA subfragment with the target nucleotide sequence are amplified and enriched ligated DNA subfragments not comprising the target nucleotide sequence.

The (amplified DNA) subfragments are sequenced, e.g., by sequencing across entire circles (long reads), PCR amplified material may also be first fragmented to create a sequencing library compatible, e.g., for ILLUMINA® or SOLiD® sequencing.

(a) Next, a contig is built from the reads, the sequences may be compared to a reference genome to identify genetic variation.

FIG. 2. Schematic showing different separations. Panel A) DNA fragments may be separated in a droplet; Panel B) DNA fragments may be separated in a microwell; Panel C) DNA fragments may be separated being bound to a DNA binding surface; and Panel D) DNA fragments may be separated being bound to a DNA binding surface on beads.

FIG. 3. Schematic showing a DNA fragment bound to a DNA binding surface. Row A) the DNA fragment is bound to the DNA binding surface via multiple binding groups—DNA fragment interactions, Row B) after fragmenting the DNA fragment, multiple fragmented DNA fragments are formed (or subfragments) that remain bound to the DNA binding surface.

### DETAILED DESCRIPTION

According to one aspect of the disclosure, a method is provided for determining the sequence of a genomic region of interest comprising a target nucleotide sequence, the method comprising the steps of, providing a DNA comprising the genomic region of interest, fragmenting the DNA to provide DNA fragments, separating the fragmented DNA, fragmenting the separated DNA fragments to provide DNA subfragments and ligating the DNA subfragments, determining at least part of the sequences of at least part of the ligated DNA subfragments that comprise the target nucleotide sequence, and using the determined sequences to build a contig of the genomic region of interest.

A “DNA,” according to the disclosure, may be derived from a DNA sample. The DNA comprises the genomic region of interest. The DNA may comprise one genome, but preferably the DNA comprises a plurality of genomes. The genomic region of interest may comprise a genomic segment on which a gene of interest resides. The genomic region of interest comprises a target nucleotide sequence. The target nucleotide sequence is a sequence that preferably is known beforehand. The target nucleotide sequence may comprise a sequence that allows the hybridization of at least one primer thereto. The target nucleotide sequence may comprise a sequence that allows the hybridization of at least two primers in an inverse orientation. Binding to the target nucleotide sequence in the genome of the two primers in a PCR reaction does not result in PCR product, only when the target nucleotide is comprised in a circularized DNA a PCR product can be formed. The target nucleotide sequence may be a sequence that allows it to be identified. For example, a target nucleotide sequence may be at least 20, 40, 60, 80, or 100 base pairs in size. The target nucleotide sequence may be at most 500, 1,000, or 2,000 base pairs in size. The size of the target nucleotide sequence may be in the range of 10-1,000, 20-500, or 30-300 base pairs in size, preferably from a non-repetitive region, this way, in the subsequent steps wherein the DNA subfragments are ligated, the DNA subfragment comprising the target nucleotide sequence can be unique, and thus the DNA subfragments ligated thereto may be representative for the genomic region of interest. Hence, in subsequent steps, i.a., in sequencing and/or amplification steps, the target nucleotide sequence may allow identifying and/or selecting the sequences representative for the genomic region of interest.

The DNA as it is provided is suitable to carry out at least the subsequent steps of fragmenting and separating. For example, a DNA sample may be subjected to lysis and/or purification steps in order to isolate the DNA from the sample to provide DNA. Hence, the DNA may be purified or may be substantially purified. The DNA may comprise genomic DNA and may be derived from multiple cells. DNA samples may be taken from a patient and/or from diseased tissue, and may also be derived from other organisms or from separate sections of the same organism, such as DNA samples from one patient, such as one DNA sample from healthy tissue and one sample from diseased tissue. DNA may be analyzed according to the methods of the disclosure and compared with a reference DNA, or different DNAs may be analyzed and compared with each other. For example, from a patient being suspected of having breast cancer, a biopsy may be obtained from the suspected tumor. Another biopsy may be obtained from non-diseased tissue. From both tissue biopsies DNA may be analyzed according to the disclosure. Hence, DNA derived from such DNA samples may be subjected to the method of the disclosure. Genomic regions of interests may, e.g., be the BRCA1 and BRCA2 gene, which genes are 83 and 86 kb long (reviewed in Mazoyer, 2005, Human Mutation 25:415-422). By determining the genomic region of interest sequence according to the disclosure, and comparing the genomic region sequences of different biopsies with each other and/or with a reference BRCA gene sequence, genetic mutations may be found that will assist in diagnosing the patient and/or determining treatment of the patient and/or predicting prognosis of disease progression. All that needs to be provided is a target nucleotide sequence in the proximity of or in the BRCA1 or BRCA2 gene.

By fragmenting the DNA, DNA fragments are provided. It is also understood that in the method of the disclosure, DNA fragments derived from a DNA may be provided, because during the processing of a DNA sample the steps of providing a DNA and the fragmenting thereof are combined. These DNA fragments are separated to provide for separated DNA fragments. The separated DNA fragments as they are now contained separately, are next further fragmented, thereby obtaining DNA subfragments, and subsequently ligated. It is understood that the fragmenting of the DNA needs to provide larger fragments as compared to the fragmenting step carried out on the separated DNA fragments.

The separation step used provides for physically separated DNA fragments that allow at least the subsequent further fragmentation and ligation step to be carried out having the DNA fragments in their physically separate state. This way, subfragments originating from the same DNA fragment can ligate with each other. The separation step may comprise separating the volume comprising the fragmented DNA over a plurality of subvolumes, wherein for each subvolume at least the steps of fragmenting to provide for DNA subfragments and the ligation of DNA fragments can be carried out. The subvolumes may be in separate containers, for example, one DNA of a fragmented DNA in a volume of 100 microliter may be divided manually over microtiter plates, e.g., in volumes of 0.5 microliter per well.

Hence, in the step of separating the DNA fragments according to the disclosure, the step of separating the DNA fragments may comprise providing each DNA fragment in a separate container. Alternatively, in the step of separating the DNA fragments according to the disclosure, the step of separating DNA fragments comprises separating the DNA fragments in portions of DNA fragments, each portion comprising several DNA fragments, preferably each portion comprising 1-2 DNA fragments and more preferably wherein each portion is in a separate container. It is understood that the number of containers over which the DNA fragments may be substantially larger than the number of DNA fragments that are provided, meaning that not all containers will comprise a DNA fragment.

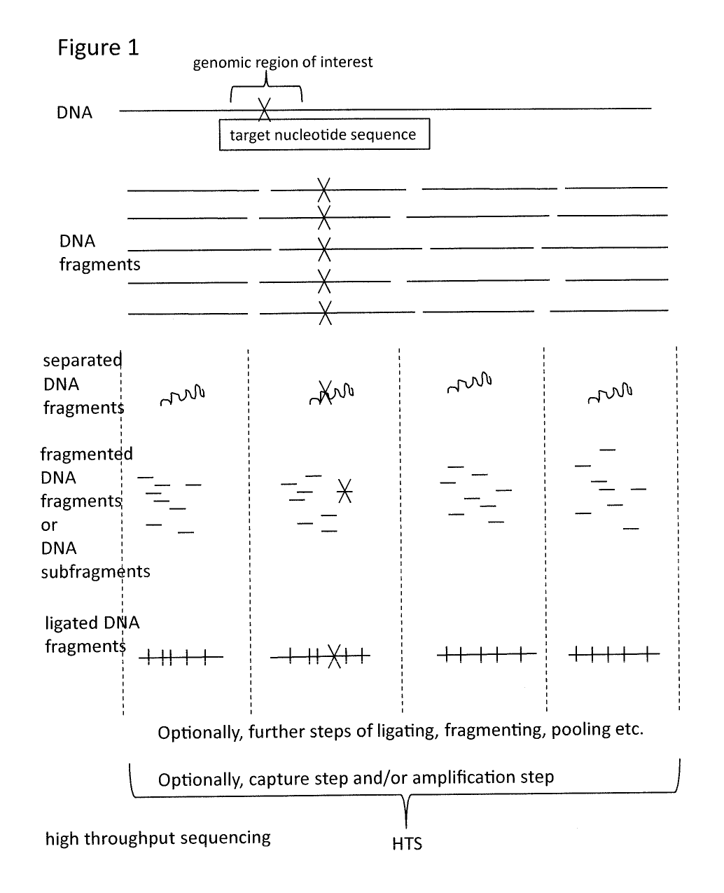

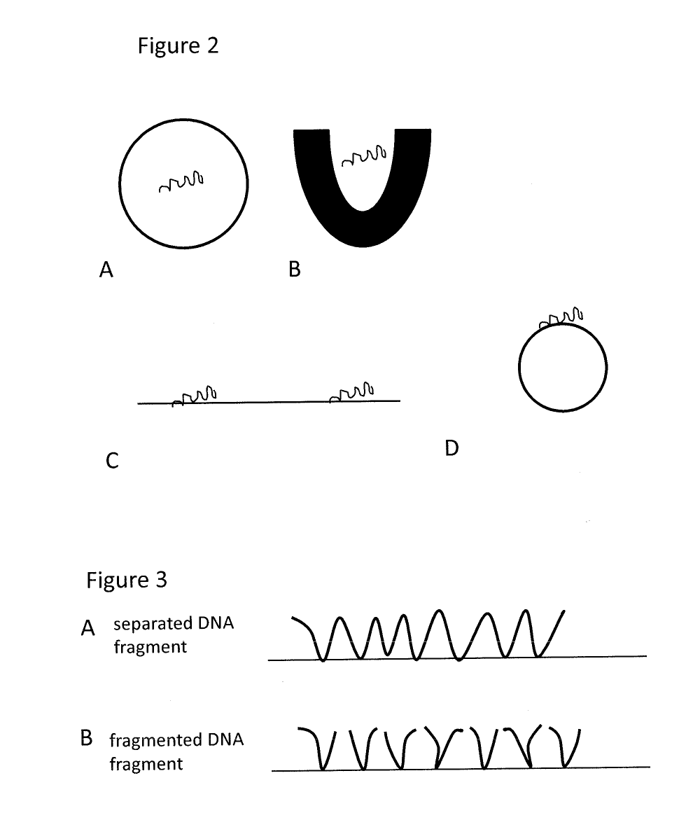

DNA fragments can also be separated by binding the DNA fragments non-covalently to a DNA binding surface (see FIG. 2). While the DNA fragments are bound to the surface, the subsequent fragmenting and ligation steps can be carried out. Binding conditions of the DNA fragments are selected such that when these fragmenting and ligation steps are carried out the DNA subfragments formed from a DNA fragments remain bound to the DNA binding surface (see FIG. 3) and also allows a ligation step to be carried out. For example, DNA fragments are bound to a DNA binding surface. The DNA binding surface with the DNA fragments can be contacted with a restriction enzyme, e.g., in an appropriate buffer that allows the enzyme to have its action and have the DNA fragments and subsequent DNA subfragments remain bound to the DNA binding surface. Next, the DNA binding surface with the DNA subfragments bound thereto can be subjected to washing steps to remove the restriction enzyme. Alternatively, DNA fragments can also be fragmented with mechanical shearing. The DNA binding surface with the DNA subfragments can next contacted with a ligation enzyme, e.g., in an appropriate buffer that allows the enzyme to have its action and have the DNA subfragments and subsequent ligated DNA subfragments remain bound to the DNA binding surface.

Hence, in one embodiment, a method is provided according to the disclosure, wherein the step of separating the DNA fragments comprises binding the DNA fragments to a DNA binding surface.

In one embodiment, a method is provided according to the disclosure, wherein the step of separating the DNA fragments comprises binding the DNA fragments to a DNA binding surface on a bead. In a further embodiment, the step of separating DNA fragments comprises separating the DNA fragments in portions of DNA fragments wherein each portion is bound to a DNA binding surface on a bead, each portion comprising several DNA fragments, preferably each portion comprising 1 or 2 DNA fragments.

DNA fragments can be, for example, copied in a single round of (linear) amplification using, e.g., random hexamer primers or long range PCR. In such an amplification, labeled nucleotides can be included, e.g., a biotin-labeled nucleotide such as Biotin-14-dCTP (19518-018) as available from Life Technologies. The concentration of such labeled nucleotide is selected such that, e.g., 1 in 1000 nucleotides of the DNA fragment will comprise the label after the single round of amplification. This DNA fragment, that is now labeled, can be used bound to a DNA binding surface via the label. For example, when the labeled DNA fragment has a biotin group, streptavidin coated beads, such as DYNABEADS® M-280 Streptavidin 11205D as available from Life Technologies, which in this embodiment is considered a DNA binding surface, can be used to bind the labeled DNA fragments. The DNA binding surface having streptavidin as DNA binding groups. Because not the whole DNA fragment is labeled, only at the labeled positions restriction enzymes and ligation enzymes may be hindered because of the binding of the label with the DNA binding group, e.g., binding of a biotin to the streptavidin group. Hence, the subsequent restriction and ligation can be carried out while the binding between the label and the DNA binding group remains. Hence, in one embodiment, the DNA binding surface comprises DNA binding group that is a ligand for a label, and the DNA fragments are provided with multiple labels, wherein the DNA fragment separating step comprises binding-labeled DNA fragments to a DNA binding group that is a ligand for the label. In a further embodiment, the labeled DNA fragments have multiple labels, and the DNA fragment is provided with a label in about 1 in 500-10,000 base pairs, or about 1 in 500-5,000 base pairs, or about 1 in 500-2,000 base pairs.

Further DNA binding surfaces are well known in the art. DNA binding surfaces are, for example, also used in DNA purification strategies and allows to bind DNA to the surface and have the DNA removed from the DNA binding surface by subjecting it, e.g., to a high salt concentration. Such DNA binding surfaces may not require labeling of the DNA fragments such as described above. DNA is negatively charged and binding surfaces that, e.g., allow anion exchange, such as anion resins, are suitable. Methods describing DNA binding surfaces used in column chromatography that may be suitable are described i.a. U.S. Pat. No. 4,935,342 A1. Preferably binding surfaces are used that bind the DNA fragments in an aqueous solution. Irrespective under which conditions the DNA is bound to the DNA binding surface, the DNA fragments, and the DNA subfragments derive therefrom preferably have to remain bound to the DNA binding surface in an aqueous solution allowing subsequent fragmenting and ligation steps to be carried out, such as, e.g., enzymatic restriction and/or ligation. For example, as described in product information for a column material used to bind nucleic acid as can be obtained from Thermo Scientific (Thermo Scientific GeneJET Stabilized and Fresh Whole Blood RNA Kit #K0871), it is described that DNA bound to a resin can be enzymatically treated with an enzyme that digests the DNA bound to the resin, likewise, DNA fragments bound to such resin may be restricted and subsequently ligated while being bound to such resin. Binding surfaces are preferred that have DNA binding groups, e.g., anion exchange groups, that can be dispersed such on the surface such that the DNA fragments when bound to the binding groups allow the DNA fragmenting step and ligation step to be carried out. Preferably, a DNA binding surface is provided wherein the binding surface is provided on beads. By using beads a large surface area can be obtained. The beads may also have cavities further enlarging the surface area, but also allowing more interaction between DNA fragments/DNA subfragments and binding groups. Beads can also be advantageous as conditions can be well controlled such that, e.g., a single DNA fragment is bound to each bead. For example, DNA fragments may be bound to silica beads such as described by Vogelstein and Gillespie in Methods Mol. Biol. 1993; 18:119-23; Isolation of DNA fragments for microinjection. Also, DNA fragments may be bound to spherical polystyrene beads with a silica surface or a carboxyl surface, e.g., DYNABEADS® Magnetic Beads as available from Life Technologies. In one embodiment, the DNA binding surface that may be a binding surface on a bead, has DNA binding groups selected from the group consisting of a DNA binding antibody, a quaternary ammonium group, a diethylaminoethyl group, a hydroxyapatite, a silicate, a borosilicate, a carboxyl.

Recently developed technologies, such as provided, for instance, by Pacific Biosciences Inc. 1380 Willow Rd. Menlo Park, Calif. 94025 and Complete Genomics, Inc., 2071 Stierlin Court, Mountain View, Calif. 94043, also provide for methods in which DNA fragments can be separated between wells on a plate on a micro scale, allowing reacting/processing single molecules (or several molecules) in a single well. Such methods can be adapted to allow the separation of the DNA fragments according to the disclosure and allow for the subsequent fragmenting and ligation step taking place inside each single well. For example, fragmenting the DNA fragments and the ligation of the DNA subfragments can take place in the same well, all that is needed is to maintain the DNA within each well while carrying out these steps. For example, a well may comprise a heat labile restriction enzyme (or DNAse), and a heat stable ligase, allowing performance of the fragmenting step or ligation step in the same well, similar to as is described in the examples. Also, the DNA fragments may be subjected to random fragmentation steps, such as sonication or shearing, simultaneously while being separated. For example, wells or droplets may be subjected to shearing and/or sonication such that the DNA fragments in the wells or droplets remains separated. For example, such technology is available and may be provided by Covaris Inc. 14 Gill Street, Unit H, Woburn, Mass., 01801-1721, USA and may be used in the methods of the disclosure. Furthermore, technologies as described in the examples and such as provided, e.g., by RainDance Technologies, Inc., 44 Hartwell Avenue, Lexington, Mass. 02421, i.e., droplet-based microfluidics technology, or as described in Williams et al., 2006, Nature Methods Vol. 3, No. 7 pages 545-550, which is incorporated herein by reference, in which millions of separate reactions (e.g., PCR) can be carried out in parallel in picoliter volumes, also provides a suitable method for separating the DNA fragments and subsequently reacting the separated DNA fragments. The DNA fragments can be divided over millions of droplets, each droplet having several DNA fragments. For example, the human genome consists of about 3×109 base pairs. When a human genome is fragmented in DNA fragments of about 100,000 base pairs, this means that about 30,000 DNA fragments are generated. The emulsion technology such as described above and in the examples, allows providing for about 108-109 droplets (or emulsion cells) per ml. This means that about 3,000-30,000 genomes may theoretically be provided as DNA fragments per ml. The droplets may comprise a heat labile restriction enzyme (or heat labile DNAse I), and a heat stable ligase, allowing performance of the fragmenting step or ligation step in the same droplet, such as is described in the examples. Alternatively, a droplet comprising a DNA fragment (or several fragments) may be subjected to subsequent fragmenting and ligation steps, e.g., by adding an enzyme (in another droplet) to each droplet, allowing the enzyme to restrict the DNA fragments in the droplets, inactivate the enzyme, and subsequently adding a ligase to each droplet (in also another droplet) and allowing the ligase to ligate the DNA subfragments in each droplet. The thus coalesced droplets may further be processed separately or may the contents may be combined and further processed. The droplet-based microfluidics technology is, i.a., described in EP2004316, which is incorporated herein by reference.

By fragmenting the separated DNA fragments, the DNA subfragments originating from a DNA fragment remain in each other's proximity because the DNA fragment is separated. When the DNA subfragments are subsequently ligated, DNA subfragments of the genomic region of interest, which are in the proximity of each other due to the separation step, are ligated. DNA subfragments comprising the target nucleotide sequence can ligate with DNA subfragments within a large linear distance on sequence level, depending on the size of the DNA fragments. For example, if the average size of DNA fragments is 50 kB and DNA fragments are generated randomly, DNA subfragments from about 50 kB on either side of the DNA subfragment carrying the target nucleotide sequence may ligate thereto, hence covering about 100 kB, provided that the DNA comprises multiple genomes. By determining (at least part of) the sequences of at least part of the ligated DNA subfragments that comprise the DNA subfragment comprising the target nucleotide sequence, sequences of DNA subfragments derived from the genomic region of interest are obtained. Each individual DNA subfragment with a target nucleotide sequence may be ligated to multiple other DNA subfragments. More than one DNA subfragment may be ligated to a DNA subfragment comprising the target nucleotide sequence. By combining at least part of the sequences of at least part of the ligated DNA subfragments, a contig of the genomic region of interest may be built.

In one embodiment of the disclosure, a method is provided for determining the sequence of a genomic region of interest comprising a target nucleotide sequence, the method comprising the steps of:
a) providing a DNA comprising the genomic region of interest;
b) fragmenting the DNA to provide DNA fragments;
c) separating the DNA fragments;
d) fragmenting the separated DNA fragments to provide for DNA subfragments;
e) ligating the DNA subfragments;
f) optionally and preferably, amplifying the ligated DNA subfragments of step e) comprising the target nucleotide sequence using at least one primer that hybridizes to the target nucleotide sequence to provide amplified DNA;
g) determining at least part of the sequences of at least part of the ligated DNA subfragments of step e) or amplified DNA of step f) comprising the target nucleotide sequences preferably using high throughput sequencing;
h) building a contig of the genomic region of interest from the determined sequences,

wherein, optionally, the ligated DNA subfragments of step e) and/or amplified DNA of step f) are, pooled.

In step a) a DNA is provided comprising the genomic region of interest. The DNA is fragmented in step b) to provide DNA fragments. The DNA fragments are subsequently separated in step c) as already outlined above. Next, the separated DNA fragments are fragmented in step d) to provide DNA subfragments. The fragmenting step b) and/or d) may comprise random fragmentation such as sonication, and may be followed by enzymatic DNA end repair. The fragmenting step may also comprise enzymatic digestion with an enzyme that cleaves the DNA at random positions, e.g., DNAse I, which in the presence of manganese ions, cleaves DNA randomly generating blunt ends and 1-2 base overhangs. In such fragmentation steps, the DNA may be repaired (enzymatically), filling in (or removing) possible 3′- or 5′-overhangs. This way DNA subfragments may be obtained in step d) that have blunt ends that allow ligation of all the DNA subfragments to adaptors and/or to each other in the subsequent step e). The fragmenting step b) and/or d) may also comprise fragmenting with one or more restriction enzymes, or combinations thereof. Fragmenting is preferably being performed controlling the fragment size. For example, by using a restriction enzyme with a specific restriction site, which is based on the statistical frequency of cleaving, allows control of the average fragment size. The fragments that are formed in step d) may have compatible overhangs or blunt ends that allow ligation of the fragments in the subsequent step e).

It is understood that the fragmenting steps b) and/or d) according to the disclosure, and similarly other fragmenting steps as well may generate a heterogeneous population of sizes. The fragmenting steps b) and/or d) may comprise a random fragmentation step. The fragmenting steps b) and/or d) may comprise fragmenting with a restriction enzyme.

The DNA fragments may be of a size of at least 10,000, 20,000, 30,000, 40,000, 50,000 100,000, 200,000 or at least 500,000. The size of the DNA fragments may be at most 150,000, 200,000, 300,000, 500,000 or at most 1,000,000 base pairs. The size of the DNA fragments may be in the range of 10,000-500,000, or 20,000-200,000, or 30,000-150,000. The DNA fragmenting step may be a step in which DNA fragments are generated with a large variety of sizes, within the ranges as listed above. Also the DNA fragments may have a large variety of sizes, and the appropriate size may be selected with a subsequent size selection step. A size selection step allows selecting the size of the DNA fragments in the ranges listed above. Size selection steps are well known in the art and may include gel extraction chromatography, gel electrophoreses or density gradient centrifugation.

The fragmenting method that is used in step d) is selected such that it results in DNA subfragments that are smaller than the DNA fragments from which they originate. In case the fragmenting step b) and d) comprise restriction enzymes, it is preferred that the restriction enzyme recognition site of step b) is longer than the recognition site of step d). This results in the restriction enzyme of step b) cutting at a lower frequency than step b), resulting in, on average, the DNA fragment in step b) being larger than the DNA subfragments.

Similar to the size ranges of the DNA fragments, DNA subfragments may be selected within an appropriate range, or the fragmenting step d) may be selected such that an appropriate size range is produced in the fragmenting step. The size of the DNA subfragments is at least 100, 200, 300, 400, 500, 750, 1,000, 1,500, or 2,000 base pairs. The size of the DNA subfragments is preferably at most 1,000, 2,000, 3,000, 5,000 or 10,000 base pairs. The size of the DNA subfragments is preferably within the range of 100-2,000 base pairs, preferably within the range of 100-1,500 base pairs.

In the next step e), the DNA subfragments are ligated. Since a DNA subfragment comprising a target nucleotide sequence may be ligated with multiple other DNA subfragments, more than one DNA subfragment may be ligated to the DNA subfragment comprising the target nucleotide sequence. This may result in combinations of DNA subfragments that are in proximity of each other as they are separated, i.e., after the separation step of the DNA fragments, at least the subsequent fragmenting and ligation step are performed having the DNA subfragments separated. This means that in the container in which the DNA fragment (or DNA fragments) may reside, the subsequent fragmenting and ligation step is carried out. Different combinations and/or order of the DNA subfragments in ligated DNA subfragments may be formed. Some of the DNA subfragments may self-ligate during this step, hence it may be advantageous to have a size separation step after the ligation step e), and separating the self-ligated DNA subfragments from the ligated DNA subfragments comprising more than one DNA subfragment. Ligation conditions may be selected that are in favor of ligating one DNA subfragment to another. For example, ligation conditions in which DNA subfragments are diluted to the extent that one DNA subfragment end rarely comes into contact with a fragment end of another DNA subfragment end are not favorable and thus not selected. In such a scenario, DNA subfragments, if possible, will preferably self-ligate. Hence, DNA subfragment concentrations are thus selected in which DNA subfragments interact such that intermolecular ligation of DNA subfragments can take place. DNA subfragments ligated to each other may be circularized in the ligation step d). Alternatively, ligated DNA subfragments may form linear molecules and not circularize in the ligation step d) because the ligation conditions are such that DNA subfragment ends may not form a closed circle. Hence, the ligated DNA subfragments may then be circularized in a separate ligation step, which may also optionally be preceded by an extra fragmenting step. In any case, appropriate conditions may be selected, which is well within the reach of the skilled person, that favor ligation reactions between DNA subfragments.

In addition to selecting favorable ligation conditions that relate to the interaction of DNA subfragments with each other, in addition, the DNA subfragment ends may be selected such that DNA subfragments may preferably ligated to another DNA subfragment instead of self-ligation. In such a scenario, DNA subfragment ends on either end of a DNA subfragment may be non-compatible, e.g., one end being blunt ended while the other end has an overhang. For example, when, for instance, a type III endonuclease would be used, which may recognize short 5-6 bp long asymmetric DNA sequences and cleave about 25-27 bp downstream thereof to leave short, single-stranded 5′ protrusions, DNA fragment ends will have either a C, T, G or A nucleotide overhang. This means that one in four nucleotide ends will be compatible and may ligate. This also means that one in four DNA subfragments can also self-ligate and may not be ligated to other DNA subfragments. Hence, building a contig may not be possible in such a scenario as there may be gaps caused by the DNA subfragments having both ends compatible, which may not end up ligated with the DNA subfragment carrying the genomic region of interest. It is also possible to perform an enzymatic restriction using at least two different restriction enzymes that provide different fragment ends in the fragmenting step d) to obtain the same effect. Hence, in these scenarios, the steps a)-e) may be performed several times each time using a different type III endonuclease, or different combination of restriction enzymes, but having steps a)-c) preferably the same, in order to obtain DNA subfragments that when combined may be sequenced and built into a contig. Alternatively, the fragmenting step may be performed suboptimally. This way, overlapping DNA subfragments may also be generated.

In addition, the fragmenting step may be a random fragmentation step, wherein also different fragment ends are generated. In such a scenario also overlapping DNA subfragments are generated. When the DNA subfragment ends are of random nature, a variety of DNA subfragment ends is generated of which not all ends are compatible. Only when ends are compatible, DNA subfragment ends will be ligated to each other. Hence, using a fragmentation method in step c) that may introduce random overhangs may be advantageous for favoring ligation of one DNA subfragment to another DNA subfragment. Such methods may be used, alone or combined with ligation conditions that also improve ligation of different DNA subfragments as already described above.

In case the DNA subfragments are obtained via enzymatic restriction, the recognition site of the restriction enzyme is known, which makes it possible to identify the DNA subfragments as remains of reconstituted restriction enzyme recognition sites may indicate the separation between different DNA subfragments. In case the DNA subfragments were obtained via random fragmentation, such as, e.g., sonication and subsequent enzymatic DNA end repair, it may be more difficult, but not impossible to distinguish one DNA subfragment from another. Irrespective of what fragmenting method is used, the ligation step e) may be performed in the presence of an adaptor, ligating adaptor sequences in between fragments. Alternatively the adaptor may be ligated in a separate step. This is advantageous because the different DNA subfragments can easily be identified by identifying the adaptor sequences that are located in between the DNA subfragments. For example, in case DNA subfragment ends were blunt ended, the adaptor sequence would be adjacent to each of the DNA subfragment ends, indicating the boundary between separate DNA subfragments.

In the next step f), optionally and preferably, the ligated DNA subfragments of step e) comprising the target nucleotide sequence are amplified using at least one oligonucleotide primer that hybridizes to the target nucleotide sequence. Such amplification may be a linear amplification.

Alternatively, when the ligated DNA subfragments have formed a circularized DNA, a primer pair may be used to amplify the ligated DNA subfragments in an inverse PCR reaction. In this way, DNA subfragments of the circularized DNA that are ligated with the DNA subfragment comprising the target nucleotide sequence, may be amplified. When the circularized DNA has a large size, it is optional to perform an additional fragmenting step of the circularized DNA and subsequently ligate again to obtain smaller circularized DNA. Smaller circularized DNA may be more suitable for an inverse PCR reaction. For example, when DNA circles are formed in step e) of 20 kB, comprising DNA subfragments of 200 base pairs, these may be digested to obtain fragments of the DNA circle of about 1000 base pairs. When these are ligated, a smaller DNA circle is formed, which comprise several DNA subfragments, which may include the DNA subfragment with the target nucleotide sequence. Performing an inverse PCR reaction on such a smaller DNA circle may be more efficient. Similarly, when linear ligated DNA subfragments are formed in step e), these may be optionally ligated such that the ends of the linear ligated DNA subfragments ligated with each other forming a DNA circle. Alternatively, in case linear ligated DNA subfragments are very large, an additional fragmenting step followed by a ligation step may be performed to generate DNA circles with an appropriate size comprising DNA subfragments, which may also be amplified in an inverse PCR reaction. As long as a circularized DNA is formed, which circularized DNA comprises DNA subfragments, such a circularized DNA may be amplified with an inverse PCR provided that it comprises the target nucleotide sequence, thereby providing amplified DNA. When an additional fragmenting step is performed before the amplification step, it is understood that the sizes of the fragmented ligated DNA subfragments are smaller than the sizes of the DNA fragment and larger than the DNA subfragments. By having the size larger than the DNA subfragments, the DNA subfragments may largely remain in the fragmented ligated DNA subfragments. The size of the fragmented ligated DNA subfragments is at least 1,000, 2,000, 3,000, 4,000, 5,000, or 6,000 base pairs. The size of the fragmented ligated DNA subfragments is preferably at most 20,000, 15,000, 12,000, or 10,000 base pairs. The size of the fragmented ligated DNA subfragments is preferably within the range of 1,000-10,000 base pairs, or within the range of 2,000-5,000 base pairs.

The amplified DNA comprises sequences corresponding to the DNA subfragments that were ligated with the DNA subfragment comprising the target nucleotide sequence. Hence, amplification is advantageous as it allows enrichment for the DNA subfragments that are derived from the DNA fragment comprising the target nucleotide sequence and hence representative of the genomic region of interest.

In one embodiment, the ligated DNA subfragments of step e) and/or amplified DNA of step f) are, pooled. Pooling of the ligated DNA subfragments of step e) may be advantageous as it allows any subsequent steps to be carried out simultaneously, instead of separately, which may be more efficient reducing the amount of reagents needed. It may be preferred to pool the ligated DNA subfragments. Furthermore, it is preferred that in any of the methods of the disclosure, an additional pooling step may be included after the step of ligating the DNA subfragments e) and before the sequencing step. However, it may also be envisaged in the methods of the disclosure to perform high throughput sequencing wherein the prepared DNA that is to be subjected to the sequencing method, e.g., the ligated DNA subfragments or amplified DNA, is still separated, i.e., the sequencing reaction may be carried out on the prepared DNA corresponding to each of the separated DNA fragments. In this embodiment, the separated DNA fragments are preferably separated such that each container comprises one DNA fragment.

Next, at least part of the sequences of at least part of the ligated DNA subfragments or the amplified DNA comprising the target nucleotide sequence is determined. Determining the sequence is preferably performed using high throughput sequencing technology, as this is more convenient and allows a high number of sequences to be determined to cover the complete genomic region of interest. From these determined sequences a contig may be built of the genomic region of interest. When sequences of the DNA subfragments are determined, overlapping reads may be obtained from which the genomic region of interest may be built. In case the DNA subfragments were obtained by random fragmentation, the random nature of the fragmentation step already may result in DNA subfragments that when sequenced results in overlapping reads. By increasing the number of genomes present in the DNA, e.g., by increasing the number of cells that are used to prepare the DNA, building a contig for the genomic region of interest may become more efficient, and reliability of the contig is improved. When the DNA subfragments were obtained with enzymes that restrict specific target sequences, multiple enzymatic reactions may be used in parallel (i.e., digesting separated DNA fragments representative of several genomes with the each of the different enzymes and subsequently ligating the DNA subfragments) in order to provide for overlapping reads. This way, overlapping reads may also be obtained. By increasing the plurality of different enzymes used, the number of overlapping fragments will increase, which may increase the reliability of the contig of the genomic region of interest that is built. Alternatively, if sequences do not overlap, e.g., when a single restriction enzyme may have been used in step d), alignment of the determined sequences with a reference sequence may also allow building a contig of the genomic region of interest.

### Multiple Target Sequences

In one embodiment, a method for determining the sequence of a genomic region of interest comprising two target nucleotide sequences is provided. This method may involve the same steps as outlined above up until the amplification step. The amplification step now uses not one target nucleotide sequence, but two. For the two target nucleotide sequences, two different primers are used in a PCR reaction, one primer for each target nucleotide sequence. When the two primer binding sites from the two target nucleotide sequences are present in ligated DNA subfragments, the two primers may amplify the sequence in between the two primer binding sites provided that the primer binding sites have the right orientation. Having ligated DNA subfragments in a circular form may be advantageous as the chance for the two primer binding sites having the right orientation is higher as compared to a linear ligated DNA subfragments (two out of four orientations will amplify, as compared to one in four for a linear ligated DNA subfragments). In a further embodiment, in addition to the two target nucleotide sequences, the genomic region of interest comprises further target nucleotides, for each target nucleotide a primer is used in the PCR amplification reaction. By combining multiple target nucleotides and corresponding primers in a single amplification will increase the chance that combinations of primers will produce an amplicon.

Hence, methods are provided for determining the sequence of a genomic region of interest according to the disclosure, wherein the genomic region of interest comprises one or more target nucleotide sequences in addition, and wherein in the amplification step a primer is provided that hybridizes with the target nucleotide sequence and one or more primers are provided for the corresponding one or more additional target nucleotides, wherein the ligated DNA subfragments are amplified, using the primers.

### Determining the Sequence of Ligated DNA Fragments

The step of determining the sequence of ligated DNA fragments preferably comprises high throughput sequencing. High throughput sequencing methods are well known in the art, and in principle any method may be contemplated to be used in the disclosure. High throughput sequencing technologies may be performed according to the manufacturer's instructions (as, e.g., provided by Roche, Illumina or Applied Biosystems). In general, sequencing adaptors may be ligated to the ligated DNA subfragments or amplified DNA. In case an amplification step is performed according to the disclosure, by using, for example, PCR as described herein, the amplified product is linear, allowing the ligation of the adaptors. Suitable ends may be provided for ligating adaptor sequences (e.g., blunt, complementary staggered ends). Alternatively, primer(s) used for PCR or other amplification method, may include adaptor sequences, such that amplified products with adaptor sequences are formed in the amplification step. In case the ligated DNA subfragments is circular and not amplified, the circularized ligated DNA subfragments may be fragmented, preferably by using, for example, a restriction enzyme in the DNA subfragment comprising the target nucleotide sequence, such that DNA subfragments ligated thereto remain intact.

Preferably long reads may be generated in the high throughput sequencing method used. Long reads may allow reading across multiple DNA subfragments of ligated DNA subfragments. This way, DNA subfragments of step d) may be identified. DNA subfragment sequences may be compared to a reference sequence and/or compared with each other. For example, as also explained hereafter, such DNA subfragment sequences may be used for determining the ratio of DNA subfragments of cells carrying a genetic mutation. By sequencing also DNA subfragment sequences of DNA subfragments adjacent to such sequences, unique ligated DNA subfragments may be identified. This is in particular the case when DNA subfragments were obtained in step d) by random fragmentation. The chance that two cells will provide for the exact same DNA subfragment is very small, let alone that the DNA subfragment ends to which such a DNA subfragment is ligated will be the same. Thus, by identifying DNA subfragments this way, the ratio of cells and/or genomic regions of interest comprising a particular mutation may be determined.

Hence, it is not required to provide for a complete sequence of the ligated DNA subfragments. It is preferred to at least sequence across (multiple) DNA subfragments, such that DNA subfragment sequences are determined.

It may also be contemplated to sequence even shorter sequences, for instance, short reads of 50-100 nucleotides. In such a scenario, it is preferred to fragment the amplified DNA or ligated DNA subfragments in smaller fragments, which may be subsequently ligated with an appropriate adaptor suitable for the high throughput sequencing method. In case a standard sequencing protocol would be used, this may mean that the information regarding the ligated DNA subfragments may be lost. With short reads it may not be possible to identify a complete DNA subfragment sequence. In case such short reads are contemplated, it may be envisioned to provide additional processing steps such that separate ligated DNA subfragments when fragmented, are ligated or equipped with identifiers, such that from the short reads, contigs may be built for the ligated DNA subfragments. Such high throughput sequencing technologies involving short sequence reads may involve paired end sequencing. By using paired end sequencing and short sequence reads, the short reads from both ends of a DNA molecule used for sequencing, which DNA molecule may comprise different DNA subfragments (corresponding to the ligated DNA subfragments), may allow coupling of DNA subfragments that were ligated. This is because two sequence reads can be coupled spanning a relatively large DNA sequence relative to the sequence that was determined from both ends. This way, contigs may be built for the amplified DNA and/or ligated DNA subfragments.

However, using short reads may be contemplated without identifying DNA subfragments, because from the short sequence reads a genomic region of interest may be built, especially when the genomic region of interest has been amplified. Information regarding DNA fragments and/or separate genomic region of interests (for instance, of a diploid cell) may be lost, but DNA mutations may still be identified.

Thus, the step of determining at least part of the sequence of at least part of the ligated DNA subfragments and/or ligated DNA may comprise short sequence reads, but preferably longer sequence reads are determined such that DNA subfragment sequences may be identified. In addition, it may also be contemplated to use different high throughput sequencing strategies for the ligated DNA subfragments and/or ligated DNA, e.g., combining short sequence reads from paired end sequencing with the ends relatively far apart with longer sequence reads, this way, contigs may be built for the genomic region of interest.

In one embodiment, the disclosure may be used to provide for quality control of generated sequence information. In the analysis of the sequences as provided by a method of high throughput sequencing, sequencing errors may occur. A sequencing error may occur, for example, during the elongation of the DNA strand, wherein the wrong (i.e., non-complementary to the template) base is incorporated in the DNA strand. A sequencing error is different from a mutation, as the original DNA that is amplified and/or sequenced would not comprise that mutation. According to the disclosure, DNA subfragment sequences may be determined, with (at least part of) sequences of DNA subfragments ligated thereto, which sequences may be unique. The uniqueness of the ligated DNA subfragments as they are formed in step d) may provide for quality control in the step of determining the sequence. When ligated DNA subfragments are amplified, and sequenced at a sufficient depth, multiple copies of the same unique (ligated) DNA subfragments will be sequenced. Sequences of copies that originate from the same ligated DNA subfragments may be compared and amplification and/or sequencing errors may be identified.

### Further Embodiments

Furthermore, according to the methods of the disclosure, from a DNA, the sequences of multiple genomic regions of interests are determined. For each genomic region of interest, a target nucleotide sequence is provided, for which corresponding primer(s) may be designed. The multiple genomic regions of interest may be genomic regions of interest that may also overlap, thereby increasing the size of which the sequence may be determined. For instance, in case a sequence of a genomic region of interest comprising a target nucleotide sequence would comprise 1 MB, combining partially overlapping genomic regions of interest, e.g., with an overlap of 0.1 MB, each with a corresponding target nucleotide sequence, combining 5 genomic regions of interest would result in a sequence of 4.6 MB (0.9+3*(0.1+0.8)+0.1+0.9=4.6 MB), thereby greatly extending the size of the genomic region of interest of which the sequence may be determined or otherwise analyzed.

Furthermore, in case a sequence of a genomic region of interest comprising a target nucleotide sequence would comprise 50 KB, combining partially overlapping genomic regions of interest, e.g., with an overlap of 5 KB, each with a corresponding target nucleotide sequence, combining five genomic regions of interest would result in a sequence of 230 KB, thereby greatly extending the size of the genomic region of interest of which the sequence may be determined or otherwise analyzed. Multiple target nucleotide sequences at defined distances within a genomic region of interest may also be used to increase the average coverage and/or the uniformity of coverage across the genomic region.

In addition, an identifier may be included in at least one of the oligonucleotide primers used in the amplification step. Identifiers may also be included in adaptor sequences; these can be used for ligation in between DNA subfragments during the ligation step d). By including an identifier in the oligonucleotide primer, when analyzing a plurality of DNAs simultaneously, the origin of each determined sequence may easily be determined. The plurality of DNAs may have been processed differently or DNAs may have been derived from samples of DNA obtained, for example, from different organisms or patients. Identifiers allow combining differently processed DNAs and/or DNAs from different origins when the processing of DNAs may converge, e.g., identical procedural steps are performed. Such convergence of processing may in particular be advantageous when the sequencing step involves high throughput sequencing.

Prior to the sequencing step, a size selection step may be performed. Such a size selection step may be performed using gel extraction chromatography, gel electrophoresis or density gradient centrifugation, which are methods generally known in the art. Preferably DNA is selected of a size between 20-20,000 base pairs, or 20,000-200,000 base pairs, preferably 50-10,000 base pairs, most preferably between 100-3,000 base pairs. A size separation step allows selecting for ligated DNA subfragments or amplified DNA in a size range that may be optimal for PCR amplification and/or optimal for the sequencing of long reads by next generation sequencing. Sequencing of reads of 500 nucleotides is currently commercially available, recent advances by companies such as the Single Molecule Real Time (SMRT™) DNA Sequencing technology developed by Pacific Biosciences (on the World Wide Web at pacificbiosciences.com/) indicate that reads of 1,000 to 10,000 nucleotides are within reach.

In case the ploidy in a cell of a genomic region of interest is greater than 1, for each ploidy a contig may be built in any one of the methods according to the disclosure. Since the genomic environment of any given target site in the genome mostly consists of DNA genome sequences that are close to the target nucleotide sequence on the linear chromosome template, because of being part of the same DNA fragment, it allows the reconstruction of each particular chromosome template. In case the ploidy of a genomic region of interest is greater than 1, multiple genomic regions of interest are present in a cell (or equivalent thereof). These multiple genomic regions of interest generally are different represented in different DNA fragments, and hence when separated are physically not in each other's proximity. When DNA fragments of such a cell are fragmented, from each genomic region of interest in a cell a corresponding DNA subfragment comprising the target nucleotide sequence will be formed. These DNA subfragments will each ligate with DNA subfragments of the corresponding DNA fragment. Ligated DNA subfragments will thus be representative of the corresponding DNA fragment and thus of the different genomic regions of interest. For instance, in case the ploidy is two, when two DNA subfragments each having a unique mutation, and separated by 1 MB, would be found together in ligated DNA subfragments, it may be concluded that these two DNA subfragments are from the same genomic region of interest. Thus, in this scenario, two DNA subfragments were identified, and are both assigned to the same genomic region. Thus, when building a contig from the sequences of identified DNA subfragments, these two DNA subfragments carrying a mutation would be used for building a contig for one particular genomic region, while the contig built for the other genomic region would not carry the mutations.

Thus, accordingly, in the methods of the disclosure, the step of building a contig may comprise the steps of:
1) identifying the DNA subfragments;
2) assigning the DNA subfragments to a genomic region; and
3) building a contig for the genomic region.

Also, when three DNA subfragments comprising a unique mutation occur (A*, B* and C*) and the ploidy of the genomic interest is two. This time, ligation products comprising two of the mutated fragments are identified, one ligation product comprising A*B* and one with A*C*. Also ligation products comprising non-mutated, DNA subfragments are identified BC and AC. In this scenario, the ligated DNA subfragments A*B and A*C* are linked by DNA subfragment A*, and ligated DNA subfragments BC and AC are linked by DNA subfragment C. In this scenario DNA subfragments A*, B* and C* are assigned to the same genomic region, while A, B and C are assigned to the other genomic region. Thus, accordingly, the step 2) may comprise of assigning the DNA subfragments to a genomic region comprises identifying the different ligated DNA subfragments of step e) and associating the different ligated DNA subfragments with the identified DNA subfragments.

Likewise, the same would apply for heterogeneous cell populations. For instance, in case a DNA is provided derived from a heterogeneous cell population (e.g., cells with different origin or cells from an organism that comprises normal cells and genetically mutated cells (e.g., cancer cells)), for each genomic region of interest corresponding to different genomic environment (which may, e.g., be different genomic environments in a cell or different genomic environments from different cells) contigs may be built.

### Identifying Mutations

In alternative embodiments, methods are provided for identifying the presence or absence of a genetic mutation, according to any of the methods above, wherein contigs are built for a plurality of DNAs, comprising the further steps of:
k) aligning the contigs of a plurality of DNAs;
l) identifying the presence or absence of a genetic mutation in the genomic regions of interest from the plurality of DNAs.

Alternatively, a method is provided according to any of the methods above, for identifying the presence or absence of a genetic mutation, comprising the further steps of:
k) aligning the contig to a reference sequence.
l) identifying the presence or absence of a genetic mutation in the genomic region of interest.

Genetic mutations can be identified, for instance, by comparing the contigs of multiple DNAs, in case one (or more) of the samples comprises a genetic mutation, this may be observed as the sequence of the contig is different when compared to the sequence of the other samples, i.e., the presence of a genetic mutation is identified. In case no sequence differences between contigs of the DNAs is observed, the absence of a genetic mutation is identified. Alternatively, a reference sequence may also be used to which the sequence of a contig may be aligned. When the sequence of the contig of the DNA is different from the sequence of the reference sequence, a genetic mutation is observed, i.e., the presence of a genetic mutation is identified. In case no sequence differences between the contig of the DNA or DNAs and the reference sequence is observed, the absence of genetic mutation is identified.

It is not required to build a contig for identifying the presence or absence of a genetic mutation. As long as sequences of ligated DNA subfragments or amplified DNA may be aligned, with each other or with a reference sequence, the presence or absence of a genetic mutation may be identified. Thus, in alternative embodiments of the disclosure, a method is provided for identifying the presence or absence of a genetic mutation, comprising the steps of the methods of the disclosure as described above, without the step of building a contig, the method comprising the further steps of:
k) aligning the determined sequences to a reference sequence.
l) identifying the presence or absence of a genetic mutation in the determined sequences.

Alternatively, a method is provided for identifying the presence or absence of a genetic mutation, comprising the steps of the methods of the disclosure as described above, without the step of building a contig, wherein of a plurality of DNAs sequences are determined, the method comprising the further steps of:
k) aligning the determined sequences of a plurality of DNAs.
l) identifying the presence or absence of a genetic mutation in the determined sequences.

### Ratio of Alleles or Cells Carrying a Genetic Mutation

As already mentioned above, when from heterogeneous cell populations DNA is provided (e.g., derived from cells with different origin or cells from an organism that comprises normal cells and genetically mutated cells (e.g., cancer cells)), for each genomic region of interest corresponding to a different genome (that may, e.g., be a different genome from different alleles in a cell or different genomes from different cells) contigs may be built. In addition, the ratio of DNA subfragments or ligated DNA subfragments carrying a genetic mutation may be determined, which may correlate to the ratio of alleles or cells carrying the genetic mutation. The ligation of DNA subfragments is a random process, the collection and order of DNA subfragments that are part of the ligated DNA subfragments may be unique and represent a single cell and/or a single genomic region from a cell. Moreover, in case the fragmenting step d) comprises a random fragmentation process, such as sonication, the points at which the DNA fragments have been broken may provide for an additional unique feature, especially within the context of the other DNA subfragments to which it is ligated (which also may have unique fragment ends).

Thus identifying ligated DNA subfragments comprising the DNA subfragment with the genetic mutation may also comprise identifying ligated DNA subfragments with a unique order and collection of DNA subfragments. The ratio of alleles or cells carrying a genetic mutation may be of importance in evaluation of therapies, e.g., in case patients are undergoing therapy for cancer. Cancer cells may carry a particular genetic mutation. The percentage of cells carrying such a mutation may be a measure for the success or failure of a therapy. In alternative embodiments, methods are provided for determining the ratio of fragments carrying a genetic mutation, and/or the ratio of ligated DNA subfragments carrying a genetic mutation. In this embodiment, a genetic mutation is defined as a particular genetic mutation or a selection of particular genetic mutations.

In a first embodiment, a method is provided for determining the ratio of fragments carrying a genetic mutation from a cell population suspected of being heterologous comprising the steps any of the methods of the disclosure as described above, without the step of building a contig, the method comprising the further steps of:
k) identifying the DNA subfragments of step d);
l) identifying the presence or absence of a genetic mutation in the DNA subfragments;
m) determining the number of DNA subfragments carrying the genetic mutation;
n) determining the number of DNA subfragments not carrying the genetic mutation;
o) calculating the ratio of DNA subfragments carrying the genetic mutation.

In an alternative embodiment, a method is provided for determining the ratio of fragments carrying a genetic mutation from a cell population suspected of being heterologous comprising the steps any of the methods of the disclosure as described above, without the step of building a contig, the method comprising the further steps of:
k) identifying the DNA subfragments of step d);
l) identifying the presence or absence of a genetic mutation in the DNA subfragments;
m) identifying the ligated DNA subfragments carrying the DNA subfragments with or without the genetic mutation;
n) determining the number of ligated DNA subfragments carrying the fragments with the genetic mutation;
o) determining the number of ligated DNA subfragments carrying the DNA subfragments without the genetic mutation;
p) calculating the ratio of ligated DNA subfragments carrying the genetic mutation.

In the methods of these embodiments, the presence or absence of a genetic mutation may be identified in step 1) by aligning to a reference sequence and/or by comparing DNA subfragment sequences of a plurality of DNAs.

In the methods according to the disclosure, an identified genetic mutation may be a SNP, single nucleotide polymorphism, an insertion, an inversion and/or a translocation. In case a deletion and/or insertion is observed, the number of fragments and/or ligation products from a sample carrying the deletion and/or insertion may be compared with a reference sample in order to identify the deletion and/or insertion. A deletion, insertion, inversion and/or translocation may also be identified based on the presence of chromosomal breakpoints in analyzed fragments.

In another embodiment, in the methods as described above, the presence or absence of methylated nucleotides is determined in DNA fragments, ligated DNA fragments, and/or genomic regions of interest. For example, the DNA of step a)-f) may be treated with bisulphite. Treatment of DNA with bisulphite converts cytosine residues to uracil, but leaves 5-methylcytosine residues unaffected. Thus, bisulphite treatment introduces specific changes in the DNA sequence that depend on the methylation status of individual cytosine residues, yielding single-nucleotide resolution information about the methylation status of a segment of DNA. By dividing samples into subsamples, wherein one of the samples is treated, and the other is not, methylated nucleotides may be identified. Alternatively, sequences from a plurality of samples treated with bisulphite may also be aligned, or a sequence from a sample treated with bisulphite may be aligned to a reference sequence.

In an alternative embodiment, in any of the methods as described herein, in the amplification step primers are used carrying a moiety, e.g., biotin, for the optional purification of (amplified) ligated DNA fragments through binding to a solid support.

In one embodiment, the ligated DNA subfragments or amplified DNA comprising the target nucleotide sequence may be captured with a hybridization probe (or capture probe) that hybridizes to a target nucleotide sequence. The hybridization probe may be attached directly to a solid support, or may comprise a moiety, e.g., biotin, to allow binding to a solid support suitable for capturing biotin moieties (e.g., beads coated with streptavidin). In any case, the ligated DNA subfragments comprising a target nucleotide sequence are captured thus allowing separation of ligated DNA subfragments or amplified DNA comprising the target nucleotide sequence from ligated DNA subfragments not comprising the target nucleotide sequence. Hence, such a capturing step allows enrichment for ligated DNA subfragments or amplified DNA comprising the target nucleotide sequence. Hence, wherein throughout the disclosure, an amplification step is performed, which is also an enrichment step, alternatively a capture step with a probe directed to the target nucleotide sequence may be performed. For a genomic region of interest at least one capture probe for a target nucleotide sequence may be used for capturing. For a genomic region of interest more than one probe may be used for multiple target nucleotide sequences.

In one embodiment, an amplification step and capture step may be combined, e.g., first performing a capture step and then an amplification step or vice versa.

In one embodiment, a capture probe may be used that hybridizes to an adaptor sequence or comprised in an amplified DNA fragment

### Example

The following example describes a method according to the disclosure and is not understood to be limiting in any way. This method is an adapted method based on the protocol for emulsion PCR as described in Williams et al., 2006, Nature Methods Vol. 3 No. 7, pages 545-550.

1. An oil-surfactant mixture is prepared by thoroughly mixing the following components at room temperature:

| Component | Amount | | Final concentration |
|---|---|---|---|
| Span 80 | 2.25 | ml | 4.5% (vol/vol) |
| TWEEN ® 80 | 200 | μl | 0.4% (vol/vol) |
| TRITON ® X-100 | 25 | μl | 0.05% (vol/vol) |
| Mineral oil to | 50 | ml | |

2. 400 μl of the oil-surfactant mixture is transferred to a vial, a stir bar is added and the mixture is stirred at 1,000 r.p.m. on a magnetic stirrer.

3. An aqueous phase solution is prepared comprising DNA fragments derived from DNA comprising a target nucleotide sequence; DNA fragments have sizes in the range of 10-100 kb. The aqueous phase comprises also a thermolabile restriction enzyme and thermostable ligase enzyme, and a buffer compatible with the restriction enzyme and ligase enzyme. The DNA comprises the genomic region of interest that comprises the target nucleotide sequence.

4. 200 μl of the aqueous phase is added dropwise to the oil-surfactant mixture over a period of 1.5 minutes. After the addition is complete, stirring is continued for 5 min. A water in oil (w/o) emulsion is thus generated comprising approximately 108 to 109 compartments (droplets) per milliliter of emulsion.

5. The mixture is incubated at 20° C. for 6 hours in order for the restriction and random religation of DNA fragments to occur.

6. The mixture is heated at 95° C. for 1 minute to inactivate the restriction enzyme.

7. The mixture is incubated at 20° C. for 6 hours for the ligase enzyme to ligate the DNA subfragments.

8. All emulsified reactions are pooled and the mixture is centrifuged at 13,000×g for 5 minutes at 25° C. The upper oil phase is disposed of.

9. Additional extractions with an organic solvent are performed to remove the remaining oil.

10. Residual solvent is removed by centrifuging under vacuum for 5 minutes at 25° C.

11. The DNA is optionally cut with a second restriction enzyme and religated with ligase to form DNA circles.

12. Circularized DNA comprising the ligated DNA subfragments are amplified with two primers specific for the target nucleotide sequence and located within one individual restriction fragment generated by the restriction enzyme used in the first restriction reaction on emulsified DNA.

13. Amplified DNA is sample prepped with a suitable Next Generation Sequencing sample preparation kit and sequenced.

14. A contig is built for the genomic region of interest based on the determined sequences.

1. A method for determining the sequence of a genomic region of interest comprising a target nucleotide sequence, the method comprising the steps of:
providing a DNA comprising the genomic region of interest;
fragmenting the DNA to provide DNA fragments;
separating the fragmented DNA;
fragmenting the separated DNA fragments to provide DNA subfragments and ligating the DNA subfragments;
determining at least part of the sequences of at least part of the ligated DNA subfragments that comprise the target nucleotide sequence; and
using the determined sequences to build a contig of the genomic region of interest.

2. A method for determining the sequence of a genomic region of interest comprising a target nucleotide sequence, the method comprising the steps of:
a) providing a DNA comprising the genomic region of interest;
b) fragmenting the DNA to provide DNA fragments;
c) separating the DNA fragments;
d) fragmenting the separated DNA fragments to provide for DNA subfragments;
e) ligating the DNA subfragments;
f) optionally and preferably, amplifying the ligated DNA subfragments of step e) comprising the target nucleotide sequence using at least one primer that hybridizes to the target nucleotide sequence to provide amplified DNA;
g) determining at least part of the sequences of at least part of the ligated DNA subfragments of step e) or amplified DNA of step f) comprising the target nucleotide sequences preferably using high throughput sequencing;
h) building a contig of the genomic region of interest from the determined sequences,
wherein, optionally, the ligated DNA subfragments of step e) and/or amplified DNA of step f) are pooled.

3. A method for determining the sequence of a genomic region of interest comprising a target nucleotide sequence, the method comprising the steps of:
a) providing a DNA comprising the genomic region of interest;
b) fragmenting the DNA;
c) separating the DNA fragments;
d) fragmenting the separated DNA fragments to provide DNA subfragments;
e) ligating the DNA subfragments;
f) fragmenting the ligated DNA subfragments of step e), preferably with a restriction enzyme;
g) ligating the fragmented ligated DNA subfragments of step f) to provide ligated DNA fragments;
h) optionally and preferably, amplifying the ligated DNA fragments of step g) comprising the target nucleotide sequence using at least one primer that hybridizes to the target nucleotide sequence to provide amplified DNA;
i) determining at least part of the sequence of at least part of the ligated DNA fragments of step g), or amplified DNA of step h) comprising the target nucleotide sequence preferably using high throughput sequencing;
j) building a contig of the genomic region of interest from the determined sequences,
wherein, optionally, the ligated DNA subfragments of step e), and/or, the fragmented ligated DNA subfragments of step f), and/or the ligated DNA fragments of step g) and/or the amplified DNA of step h) are pooled.

4. The method for determining the sequence of a genomic region of interest according to claim 2, wherein:
the genomic region of interest comprises, in addition to the target nucleotide sequence, one or more additional target nucleotide sequences, and
wherein, in the amplification step for the target nucleotide sequences, at least one primer is provided that hybridizes with the target nucleotide sequence and for each of the one or more additional target nucleotide sequences at least one primer is provided that hybridizes to the corresponding additional target nucleotide sequence.

5. The method according to claim 2, wherein the fragmenting step b) comprises a random fragmentation step.

6. The method according to claim 4, wherein the fragmenting step b) comprises fragmenting with a restriction enzyme.

7. The method according to claim 6, wherein after the fragmenting step b), a size selection step is performed.

8. The method according to claim 7, wherein the size selection step is performed using gel extraction chromatography, gel electrophoresis or density gradient centrifugation.

9. (canceled)

10. The method according to claim 1, wherein the step of separating the DNA fragments comprises providing each DNA fragment in a separate container.

11. The method according to claim 1, wherein the step of separating DNA fragments comprises separating the DNA fragments in portions of DNA fragments, each portion comprising several DNA fragments, preferably each portion comprising 1-2 DNA fragments and more preferably wherein each portion is in a separate container.

12. The method according to claim 1, wherein the step of separating the DNA fragments comprises binding the DNA fragments to a DNA binding surface.

13. The method according to claim 1, wherein the step of separating the DNA fragments comprises binding the DNA fragments to a DNA binding surface on a bead.

14. The method according to claim 13, wherein the step of separating DNA fragments comprises separating the DNA fragments in portions of DNA fragments wherein each portion is bound to a DNA binding surface on a bead, each portion comprising several DNA fragments, preferably each portion comprising 1 or 2 DNA fragments.

15. The method according to claim 2, wherein the fragmenting step d) comprises random fragmentation.

16. The method according to claim 2, wherein the fragmenting step d) comprises fragmenting with a restriction enzyme.

17. The method according to claim 2, wherein any one of the ligation steps is performed in the presence of an adaptor, and wherein preferably the ligation step e) is performed in the presence of an adaptor and the adaptor is ligated in between the DNA subfragments, and wherein, optionally, the adaptor includes an identifier.

18. (canceled)

19. (canceled)

20. The method according to claim 1, wherein the sequences of multiple genomic regions of interests are determined.

21. (canceled)

22. (canceled)

23. The method according to claim 1, wherein in case the ploidy in a cell of a genomic region of interest is greater than 1, in the step of building a contig of the genomic region of interest, a contig is built for each ploidy.

24. The method according to claim 1, wherein the step of building a contig comprises the steps of:
1) identifying the DNA subfragments;
2) assigning the DNA subfragments to a genomic region; and
3) building a contig for the genomic region.

25. The method according to claim 24, wherein the step 2) of assigning the DNA subfragments to a genomic region comprises identifying the different ligated DNA subfragments of step e) and associating the different ligated DNA subfragments with the identified DNA subfragments.

26. The method according to claim 1 for identifying the presence or absence of a genetic mutation, wherein contigs are built for a plurality of DNAs, comprising the further steps of:
k) aligning the contigs of a plurality of DNAs; and
l) identifying the presence or absence of a genetic mutation in the genomic regions of interest from the plurality of DNAs.

27. The method according to claim 1 for identifying the presence or absence of a genetic mutation, comprising the further steps of:
k) aligning the contig to a reference sequence; and
l) identifying the presence or absence of a genetic mutation in the genomic region of interest.

28.-35. (canceled)

36. The method according to claim 1, wherein the method is for determining the presence or absence of methylated nucleotides in the genomic regions of interest.
